(19) United States
(12) Patent Application Publication
(10) Pub. No.: US 2015/0117056 A1
(43) Pub. Date: Apr. 30, 2015

(54) AROMA MACHINE USING REPLACEABLE AROMA CONTAINER

(71) Applicant: Ming Jen Hsiao, Miaoli County (TW)

(72) Inventor: Ming Jen Hsiao, Miaoli County (TW)

(21) Appl. No.: 14/589,098

(22) Filed: Jan. 5, 2015

Related U.S. Application Data
(63) Continuation-in-part of application No. 14/157,302, filed on Jan. 16, 2014.
Application No. 14/157,302 is a continuation-in-part of application No. 14/042,162, filed on Sep. 30, 2013.
Application No. 14/042,162 is a continuation-in-part of application No. 13/658,820, filed on Oct. 24, 2012.

(51) Int. Cl. A61L 9/03; F21V 33/00; F21V 8/00

(52) U.S. Cl. CPC A61L 9/03; G02B 6/0045; F21V 33/004; G02B 6/0083; F21Y 2101/02
USPC 362/611

(57) ABSTRACT

An aroma machine that uses a replaceable aroma container is provided. The aroma machine includes a housing, a top cap, and a light-guiding accommodation unit coupled between the housing and the top cap. The light-guiding accommodation unit serves as a basic limiting component for the production of the aroma machine, for the components to be assembled quickly and correctly. The replaceable aroma container is placed in light-guiding accommodation unit, and is heated to release fragrance. The aroma container is disposable, or recycled. It is easy to use and replace the aroma. The light-guiding accommodation unit is made of a transparent material. Light emitted by a light emitting device in the aroma machine is guided to the light-guiding accommodation unit, and forms light decoration on an outside of the aroma machine.

### CROSS-REFERENCE TO RELATED APPLICATIONS

The present invention is a continuation-in-part of patent application Ser. No. 13/658,820 filed Oct. 24, 2012, Ser. No. 14/042,162 filed Sep. 30, 2013, and Ser. No. 14/157,302 filed Jan. 16, 2014.

### BACKGROUND OF THE INVENTION

1. Field of the Invention

This invention relates to aroma machines that heat aroma, and, more particularly, to an aroma machine that uses a disposable aroma container.

2. Description of Related Art

Currently, a heating aroma machine, such as those disclosed in U.S. Pat. No. 8,066,420, U.S. Pat. No. 8,262,277 and US2012/0024837A1, has a power source and a heating source. It is not safe for the aroma machine to use aroma or essential oil, since the aroma and the essential are likely flipped over. It also troublesome to clean the aroma. Besides, it is inconvenient to replace the aroma. If the space for the aroma to be placed therein is not cleaned completely, a new aroma with different scent from the old one will release scent blended with the old one. The accommodation space is made of a fragile material, such as ceramic and glass, and is easy to be broken when it is washed. The conventional aroma machine emits light through holes on a housing. A lamp cover is made of a transparent material. These conventional aroma machines need redesigned.

In U.S. application Ser. No. 13/658,820, light is emitted from a top cap or through holes. A periphery of an accommodation unit is included in the top cap and the inner side of the housing, without extending to a region outside of the top cap and the housing. However, the light guiding decoration between the top cap and the housing is not obvious. Besides, the accommodation unit, when assembled, is likely to be fell into the housing, and affects other components to be disposed to their correct positions. The circuit board of the light emitting diode circuit board is not protected by an insulator. The circuit board and the LED, when heated, are likely to be damaged, if not protected by an insulative cover.

### SUMMARY OF THE INVENTION

In order to solve the above problems, the present invention provides an aroma machine that uses a replaceable aroma container. An aroma and a container for the aroma can be replaced conveniently. The aroma machine has an excellent light decoration, and can be used as a night lamp. The components in the aroma machine can be assembled quickly and correctly. An insulative protection cap is further provided for prevent the aroma machine from being damaged.

The aroma machine having a replaceable aroma container comprises: a housing; a heat generating body electrically connected to a power source; a supporting member having a top side coupled to a lower side of the heat generating body and a bottom side coupled in the housing; a light-guiding accommodation unit having a heat conductive unit, an accommodation hole, a positioning flange and an extension segment light guiding ring, wherein the positioning flange is disposed outside the accommodation hole and protrudes vertically from a surface of the light-guiding accommodation unit outward, the extension segment light guiding ring extends horizontally from one side of the positioning flange, the extension segment light guiding ring has a lower side coupled to the housing, the heat conductive unit is coupled to the accommodation hole, the heat conductive unit has one side in contact with an upper side of the heat generating body, and the heat generating body heats the heat conductive unit; a top cap coupled to an upper side of the extension segment light guiding ring, whereby the extension segment light guiding ring of the light-guiding accommodation unit being fixed between the top cap and the housing, and the positioning flange being positioned on the top cap and an inner side of the housing; and a light emitting device coupled in the housing and electrically connected to a power source.

The light-guiding accommodation unit receives light emitted by the light emitting device and guides and extends the light to the extension segment light guiding ring, such that a decorative halo effect is generated at an external side of aroma. The aroma machine according to the present invention and the convention aroma machine generate different light effects. The heat generating body heats the heat conductive unit. An aroma capsule is placed in the accommodation hole and contacts with the heat conductive unit, to be heated and generate fragrance.

The top cap such as a ceramic top cap, if hit or displaced due to the external force, is limited by the flange, and is not likely to be moved or damaged.

An extension segment light guiding ring of the light-guiding accommodation unit if fixed between the top cap and the housing. A positioning flange on a lower side of the light-guiding accommodation unit is positioned inside the housing. A supporting member coupled to the light-guiding accommodation unit inside the housing and corresponding assembled heat generating body, circuit board and heat conductive unit, when assembled, are disposed on an upper edge of the extension segment light guiding ring, and the positioning flange is limited inside the housing correctly. Therefore, the light-guiding accommodation unit is limited to a correct assembly position, and will not move leftward or rightward, or be fell downward, which will affect the assembly positions of other components. Every time the aroma machine is manufactured, the light-guiding accommodation unit is used to measure the connection of the supporting member, the light emitting device and the heat generating body inside the housing. The light-guiding accommodation unit provides a fool-proofing effect, and ensures that the components can be assembled quickly and the aroma machine has a low cost.

### BRIEF DESCRIPTION OF DRAWINGS

The invention can be more fully understood by reading the following detailed description of the preferred embodiments, with reference made to the accompanying drawings, wherein:

FIG. 1 is a schematic diagram of an aroma machine that uses a replaceable aroma container according to the present invention;

FIG. 2 is an exploded view of the aroma machine shown in FIG. 1;

FIG. 3 is a cross-sectional view of the aroma machine shown in FIG. 1;

FIG. 4 is a cross-sectional view of the aroma machine shown in FIG. 1; and

FIG. 5 is a schematic diagram of the aroma machine shown in FIG. 1.

### DETAILED DESCRIPTION OF THE INVENTION

The following illustrative embodiments are provided to illustrate the disclosure of the present invention, these and other advantages and effects can be apparently understood by those in the art after reading the disclosure of this specification. The present invention can also be performed or applied by other different embodiments. The details of the specification may be on the basis of different points and applications, and numerous modifications and variations can be devised without departing from the spirit of the present invention.

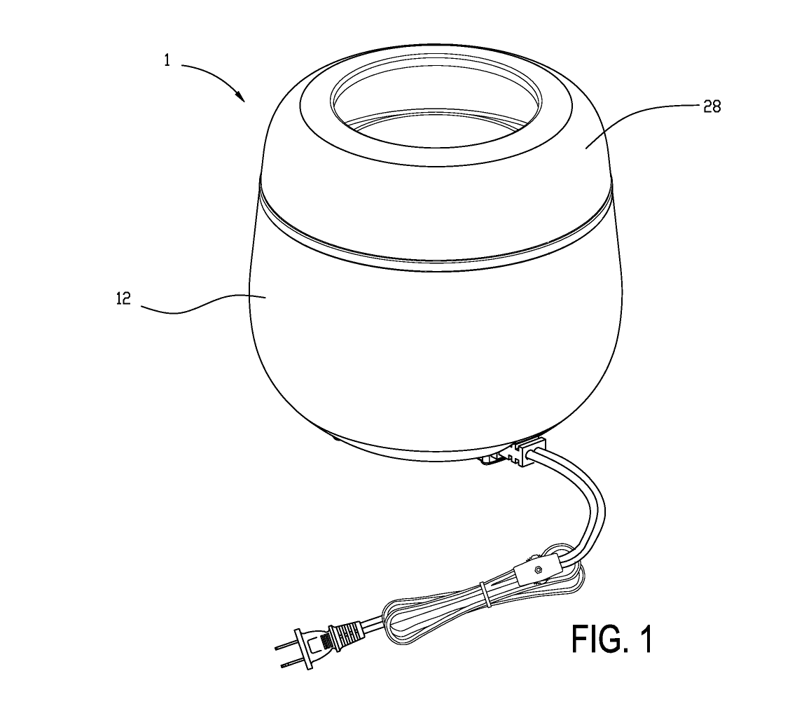

Please refer to FIGS. 1-4, which are schematic, exploded and cross-sectional views of an aroma machine 1 that uses a replaceable aroma container of a first embodiment according to the present invention.

The aroma machine 1 comprises a housing 12; a supporting member 18 having a top side 182 and a bottom side 181 coupled to the housing 1; a heat generating body 22, the supporting member 18 having a top side 182 coupled to a lower side of the heat generating body 22; a light-guiding accommodation unit 24 having a heat conductive unit 242, an accommodation hole 240, a positioning flange 245 and an extension segment light guiding ring 247, wherein the positioning flange 245 is disposed outside the accommodation hole 240 and protrudes vertically from a surface of the light-guiding accommodation unit 24 outward, the extension segment light guiding ring 247 extends horizontally from the positioning flange 245, the extension segment light guiding ring 247 has a lower side coupled to the housing 12, the heat conductive unit 242 is coupled to the accommodation hole 240, and the heat conductive unit 242 has one side in contact with an upper side of the heat generating body 22; a top cap 28 coupled to an upper side of the extension segment light guiding ring 247, wherein the extension segment light guiding ring 247 of the light-guiding accommodation unit 24 is fixed between the top cap 28 and the housing 12, and the positioning flange 245 is positioned at the top cap 28 and the inside of the housing 12; and a light emitting device 3 coupled to the housing 12.

The light emitting device 3 and the heat generating body 22 are electrically connected to a power source 4. The light-guiding accommodation unit 24 receives light emitted by the light emitting device 3, and guides the light to the extension segment light guiding ring 247, and generates a halo effect on the outside of the aroma machine 1. The heat generating body 22 heats the heat conductive unit 242.

In an embodiment, the extension segment light guiding ring 247 of the light-guiding accommodation unit 24 is fixed between the top cap 28 and the housing 12, and the positioning flange 245 on an upper side of the light-guiding accommodation unit 24 is positioned at an inside of the top cap 28. The top cap 28, such as a ceramic or glass top cap, if hit or displaced due to the external force, is limited by the positioning flange 245, and is not likely to be moved or damaged. Preferably, the positioning flange 245 is a ring-shaped flange, which has a satisfied positioning effect and is not easy to be fell.

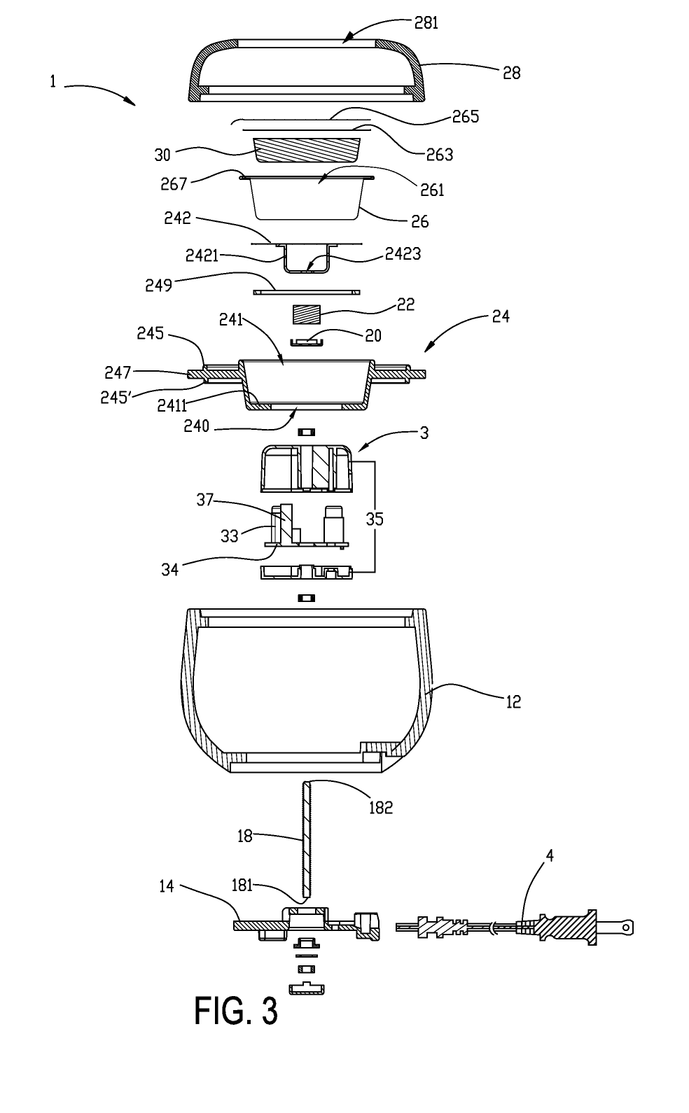

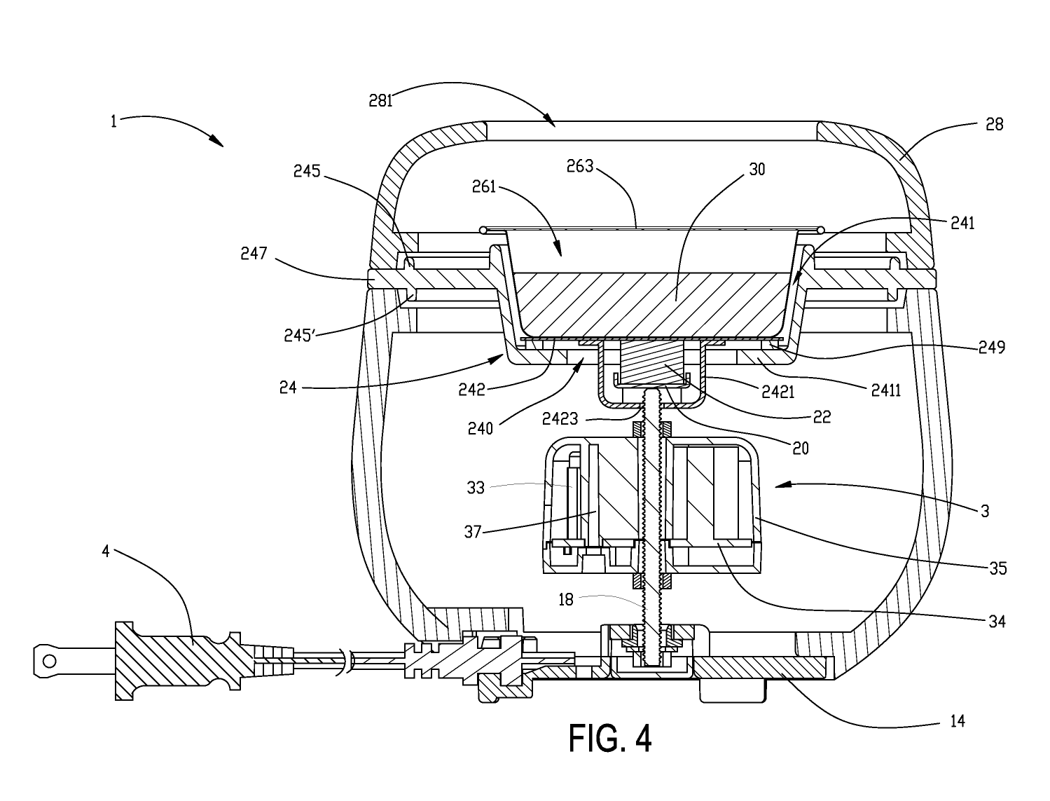

Please refer to FIGS. 3 and 4. In an embodiment the extension segment light guiding ring 247 of the light-guiding accommodation unit 24 is fixed between the top cap 28 and the housing 12, and a positioning flange 245′ on a lower side of the light-guiding accommodation unit 24 is fixed inside the housing 12. Therefore, when the supporting member 18 coupled to the light-guiding accommodation unit 24 inside the housing 12 and corresponding assembled heat generating body 22, light emitting device 3 and heat conductive unit 242 are assembled, the extension segment light guiding ring 247 is disposed on an upper edge of the housing 12, and the positioning flange 245′ on the lower side of the light-guiding accommodation unit 24 is limited and fixed to the inside of the housing 12 correctly. Therefore, the light-guiding accommodation unit 24 is limited to a correct assembly position, and will not move leftward or rightward, or be fell downward, which will affect the assembly positions of other components. Every time the aroma machine 1 is manufactured, the light-guiding accommodation unit 24 is used to measure the connection of the supporting member 18, the light emitting device 3 and the heat generating body 22 inside the housing. The light-guiding accommodation unit 24 provides a fool-proofing effect, and ensures that the components can be assembled quickly and the aroma machine has a low cost.

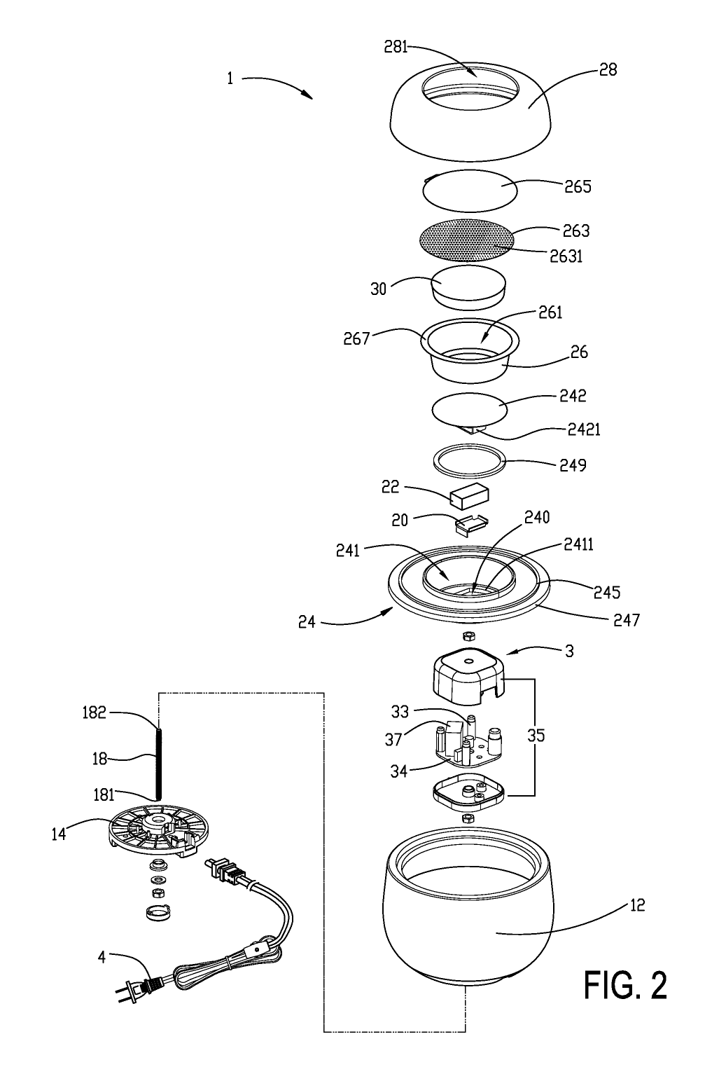

Please refer to FIGS. 2-4. In an embodiment, the light emitting device 3 comprises a light emitting body 33, a circuit board 34, an insulative transparent cover 35, and a transforming-rectifying device 37. The light emitting body 33 is electrically connected to the circuit board 34. The light emitting body 33 and the circuit board 34 are disposed in the insulative transparent cover 35. In an embodiment, the light emitting body is a light emitting diode (LED). The circuit board 34 is connected to a—power source that provides power to the light emitting body (LED). The light-guiding accommodation unit 24 receives the light emitted by the light emitting device 3, and guides the light to the extension segment light guiding ring 247. The circuit board 34 controls the LED to flash or change color, in order for the aroma machine 1 according to the present invention to provide a halo effect, which is different from the conventional aroma machine.

The transforming-rectifying device 37 is disposed in the aroma machine 1 and the insulative transparent cover 35 to be protected. The transforming-rectifying device 37 is electrically connected to the circuit board 34. In an embodiment, the transforming-rectifying device 37 is a capacitor or a rectifier, for reducing an alternating voltage AC and transforms the alternative voltage AC into a direct voltage DC, for being used by the present invention. In an embodiment, a power plug 4 does not to be big or has a transforming rectifying device 37, and is easy for storage.

Please refer to FIGS. 2-4. In an embodiment, the accommodation hole 240 comprises an accommodation space 241, and an inner flange 2411 disposed on a bottom side of the accommodation space 241. The heat conductive unit 242 is coupled to the inner flange 2411. A heating structure is specially designed to the aroma machine that uses a replaceable aroma container 26 according to the present invention. In an embodiment, the heat conductive unit 242 is a metal piece. The metal piece, if heated the heat generating body 22, conducts and spreads heat, and reduces the temperature. The replaceable aroma container 26 is heated, and releases fragrance.

Please refer to FIGS. 2-4. In an embodiment, the heat conductive unit 242 further comprises a screw hole rack 2421 disposed on a bottom side of the heat conductive unit 242. The screw hole rack 2421 comprises a fixture hole 2423. The aroma machine 1 further comprises a protection cap 20, and a bottom cap 14 coupled to the other side (bottom side) of the housing 12. When manufactured and assembled, the extension segment light guiding ring 247 is disposed on an upper side of the housing 12, and the positioning flange 245′ is limited and fixed to the inside of housing 12 correctly. Therefore, the light-guiding accommodation unit 24 is limited to a correct assembly position, and will not move leftward or rightward, or be fell downward, which will affect the assembly positions of other components. The light-guiding accommodation unit 24 determines the coupling of the screw hole rack 2421 of the heat conductive unit 242 to accommodation hole 240. The heat generating body 22 has one side against the heat conductive unit 242. The bottom side of the supporting member 18 penetrates through the light emitting device 3. The circuit board 34 is coupled to the bottom cap 14. The top side of the supporting member 18 penetrates into the fixture hole 2423 of the screw hole rack 2421. The protection cap 20 is coupled between the supporting member 18 and heat generating body 22. The protection cap 20 and the heat generating body 22 are disposed in a space formed by the screw hole rack 2421, so as to correctly and quickly assemble the components.

Please refer to FIGS. 2-4. In an embodiment, the light-guiding accommodation unit 24 further comprises a heat insulation pad 249 coupled between the inner flange 2411 and the heat conductive unit 242, for preventing the heat generating body 3 from conducting the heat conducted to the heat conductive unit 24 further to the light-guiding accommodation unit 24, Therefore, the light-guiding accommodation unit 24 is protected from damage and has a longer life.

In an embodiment, the light-guiding accommodation unit 24 is transparent or semi-transparent plastic, silicone, acrylic, glass, or transparent ceramic.

In an embodiment, the heat generating body 22 is a resistor, such as a PTC, a thermal sensitive resistor, or a cement resistor. In an embodiment, it is the PTC resistor that is selected, which can be heated constantly. Therefore, when the power source provides power, the PTC generates heat that melts or vaporize the aroma 30, which then releases fragrance.

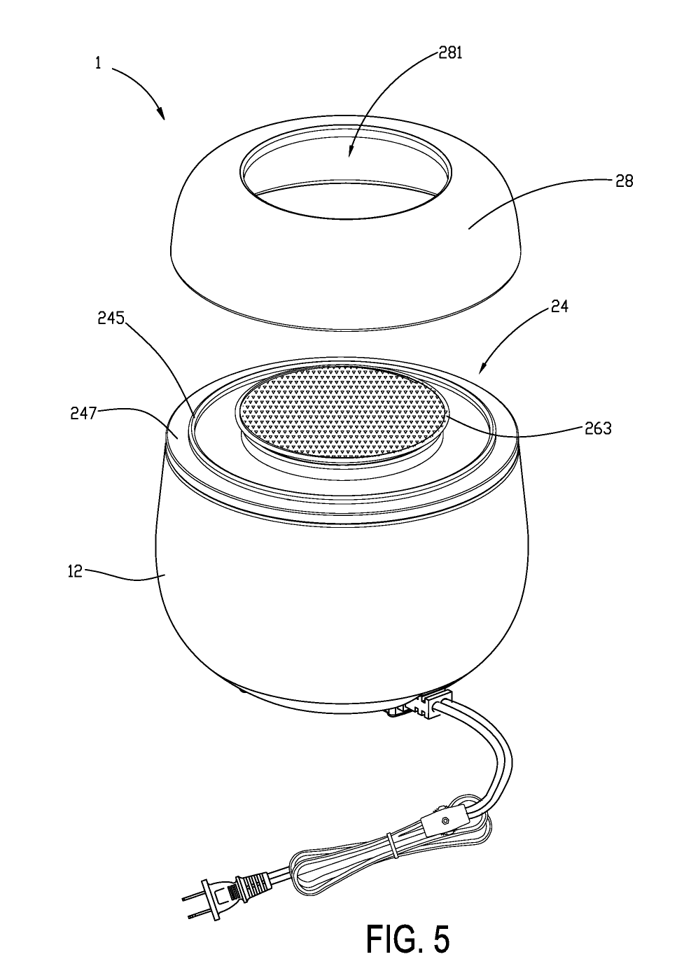

Please refer to FIGS. 2-5. In an embodiment, the replaceable aroma container 26 further comprises a replaceable aroma container 26 disposed in the light-guiding accommodation unit 24. The replaceable aroma container 26 has an aroma 30 contained therein. In an embodiment the aroma 30 is scent wax. The replaceable aroma container 26 is in contact with the other side of the heat conductive unit 242 and heated up to generate the aroma. After the aroma releases fragrance completely, the replaceable aroma container 26 can be disposed or recycled, which is different from the use of the aroma of the conventional aroma machine.

In an embodiment, the replaceable aroma container 26 further comprises a ventilation film 263, and the ventilation film 263 comprises a ventilation hole 2631. The ventilation film 263 covers the opening 261 of the replaceable aroma container 26, and blocks the aroma 30 in the accommodation trough of the replaceable aroma container 26.

In an embodiment, the ventilation film 263 is cloth, fiber piece, through hole resin film or through hole metal foil.

In an embodiment, the replaceable aroma container 26 is an aluminum foil bowl, and the replaceable aroma container of the aluminum foil bowl further comprises a periphery 267. The periphery 267 is formed by extending horizontally from one side of the opening 261. The ventilation film 263 is coupled to the periphery 267 that is extends horizontally from one side of the opening 261.

The foregoing descriptions of the detailed embodiments are only illustrated to disclose the features and functions of the present invention and not restrictive of the scope of the present invention. It should be understood to those in the art that all modifications and variations according to the spirit and principle in the disclosure of the present invention should fall within the scope of the appended claims.

What is claimed is:

1. An aroma machine having a replaceable aroma container, comprising:
a housing;
a heat generating body electrically connected to a power source;
a supporting member having a top side coupled to a lower side of the heat generating body and a bottom side coupled in the housing;
a light-guiding accommodation unit having a heat conductive unit, an accommodation hole, a positioning flange and an extension segment light guiding ring, wherein the positioning flange is disposed outside the accommodation hole and protrudes vertically from a surface of the light-guiding accommodation unit outward, the extension segment light guiding ring extends horizontally from one side of the positioning flange, the extension segment light guiding ring has a lower side coupled to the housing, the heat conductive unit is coupled to the accommodation hole, the heat conductive unit has one side in contact with an upper side of the heat generating body, and the heat generating body heats the heat conductive unit;
a top cap coupled to an upper side of the extension segment light guiding ring, whereby the extension segment light guiding ring of the light-guiding accommodation unit being fixed between the top cap and the housing, and the positioning flange being positioned on the top cap and an inner side of the housing; and
a light emitting device coupled in the housing and electrically connected to a power source;
wherein the light-guiding accommodation unit receives light emitted by the light emitting device and guides and extends the light to the extension segment light guiding ring, such that a decorative halo effect is generated at an external side of aroma.

2. The aroma machine of claim 1, wherein the light emitting device comprises an insulative transparent cover, a light emitting body disposed in the insulative transparent cover, a circuit board disposed in the insulative transparent cover and electrically connected to the power source and the light emitting body, a transforming-rectifying device electrically connected to the circuit board.

3. The aroma machine of claim 1, wherein the heat conductive unit further comprising a screw hole rack disposed on a bottom side of the heat conductive unit bottom side and having a fixture hole and a bottom cap coupled to a lower side of the housing, the screw hole rack of the heat conductive unit is coupled to the accommodation hole, the heat generating body has one side against the heat conductive unit, the bottom side of the supporting member is coupled to the bottom cap, and the top side of the supporting member top side penetrates through a fixture hole of the screw hole rack.

4. The aroma machine of claim 3, wherein the light-guiding accommodation unit has an inner flange extending horizontally in the accommodation hole and coupled to the heat conductive unit.

5. The aroma machine of claim 4, wherein the light-guiding accommodation unit further comprises a heat insulation pad coupled between the inner flange and the heat conductive unit.

6. The aroma machine of claim 1, wherein the heat generating body is a resistor.

7. The aroma machine of claim 1, wherein the aroma machine further comprises a protection cap coupled between the heat generating body and the supporting member.

8. The aroma machine of claim 6, wherein the aroma machine further comprises a protection cap coupled between the heat generating body and the supporting member.

9. The aroma machine of claim 1, wherein the aroma machine further comprises a replaceable aroma container disposed in the accommodation hole of the light-guiding accommodation unit, for an aroma to be placed therein, the replaceable aroma container being in contact with another side of the heat conductive unit.

10. The aroma machine of claim 3, wherein the aroma machine further comprises a replaceable aroma container disposed in the accommodation hole of the light-guiding accommodation unit, for an aroma to be placed therein, the replaceable aroma container being in contact with another side of the heat conductive unit.

11. The aroma machine of claim 4, wherein the aroma machine further comprises a replaceable aroma container disposed in the accommodation hole of the light-guiding accommodation unit, for an aroma to be placed therein, the replaceable aroma container being in contact with another side of the heat conductive unit.

12. The aroma machine of claim 9, wherein the replaceable aroma container further comprises a ventilation film that has a ventilation hole, and covers the opening of the replaceable aroma container to block the aroma in an accommodation trough of the replaceable aroma container.

13. The aroma machine of claim 12, wherein the ventilation film is cloth, fiber piece, through hole resin film or through hole metal foil.
